(19) United States
(12) Patent Application Publication
(10) Pub. No.: US 2024/0066097 A1
(43) Pub. Date: Feb. 29, 2024

(54) COMPOSITION FOR PATTERN RECOGNITION BASED TARGETING AND ACTIVATING AN INNATE IMMUNE RESPONSE

(71) Applicant: Phyto42, Las Vegas, NV (US)

(72) Inventors: Kevin Lau, San Jose, CA (US); Lawrence Chan, Sunnyvale, CA (US)

(21) Appl. No.: 18/238,641

(22) Filed: Aug. 28, 2023

Related U.S. Application Data
(60) Provisional application No. 63/446,407, filed on Feb. 17, 2023.
(60) Provisional application No. 63/402,278, filed on Aug. 30, 2022.

(51) Int. Cl. A61K 38/16; A61K 31/122; A61K 31/352; A61K 31/353

(52) U.S. Cl. CPC A61K 38/168; A61K 31/122; A61K 31/352; A61K 31/353

(57) ABSTRACT

Embodiments of a composition provide a line of defense against SARS coronaviruses that complements vaccines. The composition may be taken orally, intravenously, nasally, or inhaled by a healthy person to prevent a SARS infection or by a sick person infected by a SARS coronavirus for treatment. The composition is configured for molecular pattern recognition based targeting of the SARS coronavirus spike (S) protein and activating an innate immune response. The composition includes a lectin component. The composition may also include an optional flavonoid component, an optional calcium component, or both.

### PRIORITY CLAIM

This application claims the benefit under 35 U.S.C. § 119(e) of provisional application 63/402,278, filed Aug. 30, 2022, and of provisional application 63/446,407, filed Feb. 17, 2023, entire contents of which are hereby incorporated herein by reference for all purposes as if fully set forth herein.

### TECHNICAL FIELD

The invention relates to methods and compositions for treating viruses, including coronaviruses such as SARS-CoV-2, its analogues, progeny and variants. More particularly, it relates to a composition for pattern recognition based targeting of a glycosylated pathogen(s), activating an innate immune response to destroy the pathogen, and inhibiting the pathogen's reproduction. The invention also relates to the use of artificial intelligence and machine learning, as applied to pharmaceutical drugs and drug discovery.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

The Severe Acute Respiratory Syndrome CoronaVirus 2 (SARS-CoV-2) virus is responsible for the global COVID-19 pandemic, which continues to spread. Public health measures in the USA and other countries have had limited efficacy with masks and social distancing as the primary methods for reducing infection rates by presenting physical barriers to reduce exposure and lower viral loads.

Vaccines, which train the human immune system to provide an acquired immunity, are another method for reducing infection rates. COVID-19 vaccines help protect against the illness by contributing to adaptive immunity and training our adaptive immune system to produce relevant antibodies. In particular, vaccines attempt to train our adaptive immune system to produce antibodies that target exposed portions of pathogens, including, for example, the spike protein of SARS coronaviruses. But, the SARS spike protein is continuously mutating, and it can be difficult for vaccines to keep up with these mutations. And, vaccines have had some difficulty in protecting against Long COVID or secondary effects. Modern vaccines are also inaccessible to many due to their high cost and required logistics. Even if vaccines were accessible to everyone, vaccine efficacy has been shown to wane over time, which means that frequent boosters, possibly matched to the current strains, are needed. There already exists vaccine hesitancy among the general population, and those who are fully vaccinated are becoming reluctant, for various reasons, to receive periodic boosters, especially with the frequency of breakthrough infections.

Accordingly, there is an urgent need for another approach against SARS coronaviruses that is effective, fast-acting, affordable, accessible, and that complements vaccines.

### SUMMARY

The appended claims may serve as a summary of the invention.

### BRIEF DESCRIPTION OF THE FIGURES

The present invention is illustrated by way of example, and not by way of limitation, in the figures of the accompanying drawings and in which like reference numerals refer to similar elements and in which:

FIG. 1 illustrates MBL binding and complement activation.

FIG. 2 illustrates a flow diagram of an immune system activation process.

FIG. 3 illustrates an example composition in accordance with some embodiments.

FIG. 4 illustrates an example data flow in accordance with some embodiments.

FIG. 5 illustrates a block diagram of a computing device in which the example embodiment(s) of the present invention may be embodiment.

FIG. 6 illustrates a block diagram of a basic software system for controlling the operation of a computing device in accordance with an embodiment.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

Embodiments are described herein in sections according to the following outline:
1.0 GENERAL OVERVIEW
2.0 HUMAN IMMUNE SYSTEM INTRODUCTION
3.0 MANNOSE BINDING LECTIN INTRODUCTION
4.0 CORONAVIRUS VARIANTS AND THEIR SIMILARITIES
5.0 MANNOSE BINDING LECTINS AND CORONAVIRUSES
6.0 PLANT LECTINS
7.0 ARTIFICIAL INTELLIGENCE AND MACHINE LEARNING
8.0 FLAVONOIDS
9.0 CALCIUM
10.0 COMPOSITION FOR PATTERN RECOGNITION BASED TARGETING AND ACTIVATING AN INNATE IMMUNE RESPONSE
   10.1 CONCENTRATION DETERMINATIONS
   10.2 EXAMPLE COMPOSITION 1
   10.3 EXAMPLE COMPOSITION 2
   10.4 EXAMPLE COMPOSITION 3
   10.5 EXAMPLE COMPOSITION 4
11.0 HARDWARE OVERVIEW
12.0 SOFTWARE OVERVIEW
13.0 OTHER ASPECTS OF DISCLOSURE

1.0 General Overview

The present disclosure is based, in part, on the unexpected discovery that certain lectins mitigate COVID-19 risks. In particular, these certain lectins are capable of inhibiting the Severe Acute Respiratory Syndrome CoronaVirus 2 (SARS-CoV-2) virus. For example, Inventor has found several plant lectins that have high affinity for SARS-CoV-2 and isolated one of those with high affinity from Allium porrum. A composition comprising one or more lectins enables an innate immune response against SARS-CoV-2, thereby reducing infection risk and improving outcomes. A composition may also include a supportive component to facilitate lectin binding. A composition may also include a flavonoid that inhibits viral replication.

In one aspect, a composition for molecular pattern recognition based targeting of one or more infectious agents and activating an innate immune response is provided. The composition includes a lectin component that binds to one or more glycosylated pathogens and that, upon binding, acts as an opsonin. The lectin component comprises at least one lectin. Lectins may be classified, for example, by their affinity where an example lectin may be one of a mannose binding lectin (MBL), a N-acetyl glucosamine binding lectin, or a jacalin binding lectin. Lectin classification continues to evolve and it's important to note that their classification does not imply any limits to the lectin binding affinity. For example, a mannose binding lectin may also have high binding affinity to N-acetyl glucosamine and an N-acetyl glucosamine lectin may also have high binding affinity for mannose. As a result, the terms mannose binding lectin and MBL may be interpreted as either the classification and/or a lectin's ability to binding to high-mannose or fucose structures as appropriate.

An example lectin may be a natural lectin isolated or extracted from a plant, such as Allium porrum, Urtica dioica, or Nicotiana tabacum. An example lectin may be a manufactured lectin synthesized and/or derived from a plant, such as Allium porrum, Urtica dioica, or Nicotiana tabacum. Lectins are proteins that may form and/or re-form into one of many structures including, most commonly: a monomer structure, a dimer structure, a trimer structure, or a tetramer structure. A lectin consisting of a single protein strand is known as a monomer. When a lectin is composed of multiple strands (e.g., dimer, trimer, tetramer, etc.), the strands may be the same or different which results in a multitude of possible lectin forms. When the strands are the same, the homo- prefix may be used to describe a lectin (e.g., homodimer, homotrimer, homotetramer, etc.) When the strands are different, the hetero- prefix may be used to describe a lectin (e.g., heterodimer, heterotrimer, heterotetramer, etc.). The structure of a lectin defines its binding affinity and binding specificity which are key factors determining the amount of lectin necessary to be effective against a glycosylated pathogen.

In addition to binding efficacy (e.g., binding constant and/or EC₅₀), binding affinity and binding specificity, an amount of a lectin necessary to be effective for use against the one or more glycosylated pathogens is also concentration dependent based on an amount of blood in an individual receiving the composition and the intended purpose. For example, 3 mg of lectin for an average adult with 5 L of blood may be approximately equivalent to 1 mg of lectin for an adolescent with 1.6 L of blood. For the purposes of this disclosure, 5 L (5,000 ml) of blood will be assumed for an average adult where appropriate unless otherwise specified as conversions are well understood. An example amount of lectin in the composition for a single dose is at least 0.25 mg (250 μg) but no more than 11.25 mg (11,250 μg), which also depends on how frequent each dose is administered and the half life for the components of the composition. Regarding the intended purpose, the amount of lectin may be dependent on whether the composition is taken for therapeutic or preventive purposes where therapeutic uses require a higher amount and concentration than preventative uses.

The composition may also include a flavonoid component that inhibits viral reproduction by, for example, binding to and inhibiting the main protease of the one or more glycosylated pathogens. The main protease of coronaviruses is required for viral replication and reproduction. Thus, inhibiting the function of the main protease inhibits viral replication and reproduction. The optional flavonoid component may comprise at least one flavonol from a class of flavonoids. Kaempferol and quercetin are two example flavonols effective at binding to and inhibiting SARS-CoV-2's main protease. The optional flavonoid component may comprise, instead of or in addition to the at least one flavonol, at least one flavanol or catechin from the class of flavonoids. EGCG (epigallocatechin gallate) is an example flavanol effective at binding to and inhibiting SARS-CoV-2's main protease. An amount of the flavonoid component may be based on one or more ratios of the flavonoid to lectin (e.g., MBL) in a plant containing a lectin effective against the SARS-CoV-2, and/or based on the amount of the lectin component in the composition. An amount of a flavonoid may also be dependent on its binding efficacy (e.g., binding constant and/or EC₅₀) independent from the amount of lectin in the composition. An example amount of a flavonoid in the composition for a single dose is between approximately 0.68 mg and approximately 30.38 mg of kaempferol. Another example amount of a flavonoid in the composition for a single dose is between approximately 0.23 mg and approximately 10.13 mg of quercetin. Another example amount of a flavonoid in the composition for a single dose is between approximately 0.19 mg and approximately 189 mg of EGCG.

The composition may also include a supportive component that facilitates the lectin component binding to the one or more glycosylated pathogens. An example supportive component is calcium. An amount of the supportive component may be based on a ratio of calcium to MBL in a plant containing a lectin effective against the SARS-CoV-2 virus and/or based on the amount of the lectin component in the composition. An example amount of calcium in the composition for a single dose may be at least 14.75 mg but no more than a typical daily dose of calcium for an adult (e.g., 625 mg).

The composition may be taken orally, intravenously, nasally, or inhaled to mitigate the chances of being infected by the one or more glycosylated pathogens, such as the SARS-CoV-2 virus, and/or to shorten the time to recover from an infection (e.g., SARS-CoV-2 infection). The composition supports an additional line of defense utilizing our innate immune system against one or more glycosylated pathogens, including SARS coronaviruses. As an illustration, an example composition may include a pharmaceutically-acceptable carrier and/or excipient for delivering the lectin component inside a patient.

Although techniques herein relate to treating coronaviruses, the techniques are applicable to other areas such as HIV, cancer, multiple sclerosis, “common” cold, influenza, etc. For example, a composition for treating one or more pathogens may include at least one of: a lectin in an amaryllidaceae family plant, a lectin in an urticaceae family plant, a lectin in a solanaceae family plant, a lectin in a bromeliaceae family plant, and a lectin in a musaceae family plant. Examples of an amaryllidaceae family plant include Allium cepa (onion), Allium cepa (shallot), Allium porrum (leek), Allium sativum (garlic), Allium schoenoprasum (chive), Haemanthus (cape tulip), Narcissus pseudonarcissus (daffodil), Narcissus jonquilla (jonquil), Galanthus (snowdrop), Leucojum (snowflake), Narcissus pseudonarcissus (daffodil). Examples of an urticaceae family plant include Urtica dioica (stinging nettle), Laportea (wood nettles), Dendrocnide (Australian stinging trees), Pilea microphylla (artillery plant), Helxine soleiroli (baby tears), and Cecropia peltata (trumpet tree). Examples of a solanaceae family plant include Brugmansia (angel's trumpet), Atropa belladonna (belladonna), Datura stramonium (jimsonweed), Physalis peruviana (cape gooseberry), Physalis philadelphia (tomatillo), Hyoscyamus niger (henbane), Mandragora (mandrake), Solanum rostratum (buffalo bur), Solanum melongena (eggplant), Solanum tuberosum (potato), Solanum lycopersicum (tomato), Solanum dulcamara (woody nightside), members of the Capsicum genus (e.g., Capsicum annum (bell pepper), Capsicum annuum (cayenne pepper), Capsicum species (chili pepper), Capsicum chinense (ghost pepper), Capsicum annuum (pimiento), Capsicum frutescens (tabasco pepper)), Petunia (petunia), and the Nicotiana genus which includes, for example, Nicotiana tabacum (tobacco). Examples of a bromeliaceae family plant include Ananas comosus (pineapple), along with members of the aechmea and bromelia genus (e.g., Aechmea antiacantha and Bromelia chrysantha). Examples of a musaceae family plant include Musa acuminata (banana), Musa balbisiana (plantain), and Musa sapientum (common banana).

Other embodiments, aspects, and features will become apparent from the reminder of the disclosure as a whole.

2.0 Human Immune System Introduction

The human immune system is a complex combination of organs, cells, proteins and tissues, which work together to protect the human body from invading pathogens. Our immune system consists of two parts: an innate immune system and an adaptive immune system.

Innate immunity is considered a general or “nonspecific” defense system. Our innate immune system (which includes our skin, mucous membranes, phagocytes, natural killer cells, and various proteins and enzymes) is our first line of defense against invading pathogens. The purpose of the innate immune response is to immediately prevent the spread and movement of invading pathogens throughout the human body by blocking their entry and generating rapid inflammatory responses in response to signals from molecular pattern recognition receptors. The innate immune system is constantly protecting us from infection by pathogens via pathogen recognition and elimination.

Adaptive immunity is our second line of defense against invading pathogens. If invading pathogens evade our innate immune system, then our adaptive immune system is activated. Our adaptive immune system builds antibodies through, for example, exposure to diseases or vaccinations. Vaccines train our adaptive immune system to provide an acquired immunity, which is activated after pathogens evade our innate immune system.

3.0 Mannose Binding Lectin Introduction

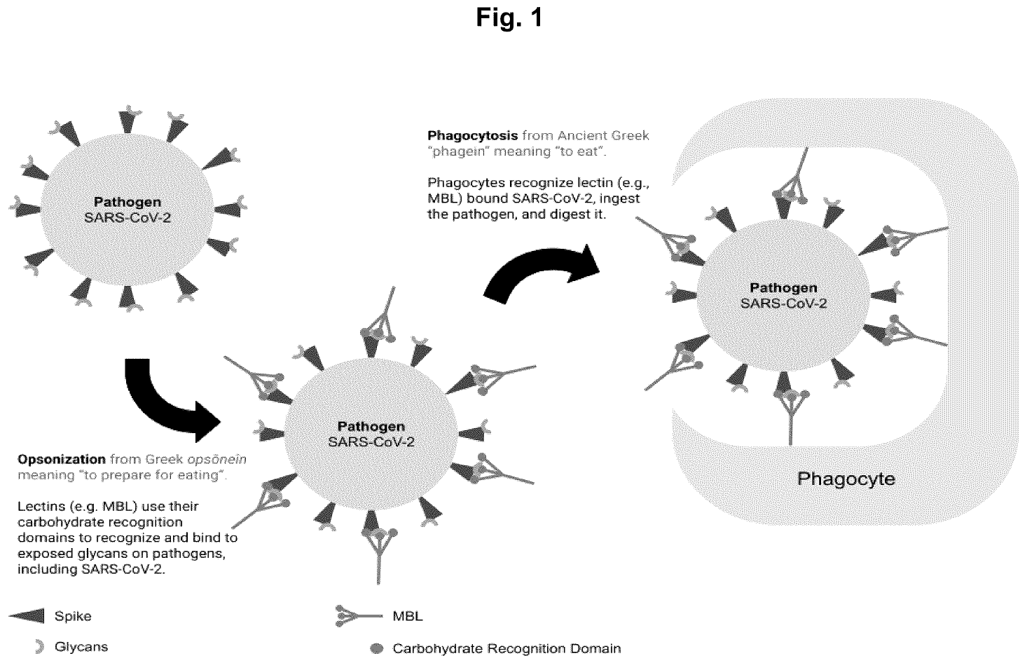

One part of the innate immune system includes lectins, specifically mannose binding lectins (MBLs), which help our innate immune system recognize pathogens. Lectins are commonly identified as an “anti-nutrient” due to their association with various negative health effects and conditions. MBLs are proteins produced by the liver and secreted into the serum where they can activate an immune response. MBLs have carbohydrate recognition domains for prototypical pattern recognition allowing them to recognize and bind to infectious agents, including bacteria and viruses, and activating the innate immune system against recognized pathogens. The carbohydrate recognition domains on MBLs recognize pathogen-associated molecular patterns (PAMPs) to identify pathogens for the innate immune system to destroy. MBL binding and complement activation enhances phagocytosis by acting as an opsonin using a lectin pathway, as illustrated in FIG. 1. Opsonization leads to phagocytosis where the bound pathogens are destroyed. Complement activation further serves as a bridge to the adaptive immune system for producing antigen specific antibodies, possibly resulting in acquired immunity.

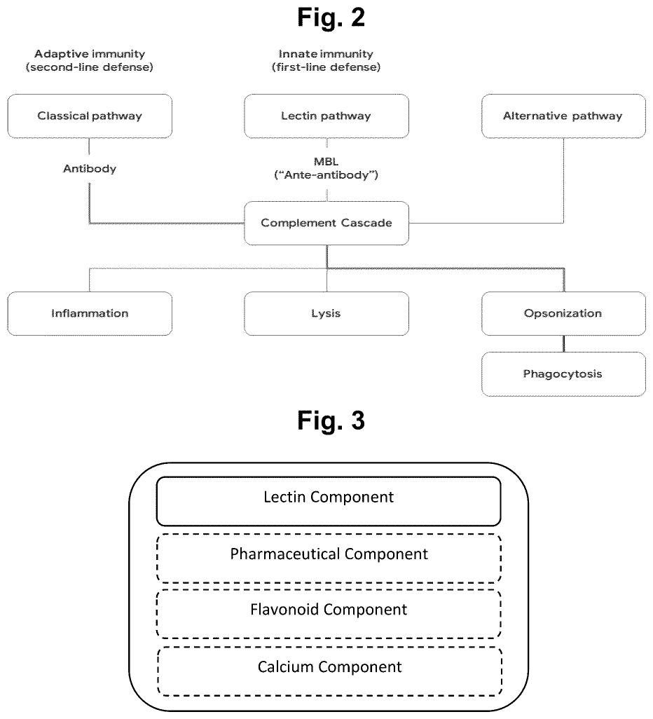

FIG. 2 illustrates a flow diagram of an immune system activation process. As part of our innate immune system, MBLs function as part of our first line of defense against invading pathogens. Conceptually, MBLs may be considered as an “ante-antibody” because of its defensive role at recognizing pathogens during the initial delay period required to develop an antibody response.

As illustrated in FIG. 2, MBLs (lectin pathway) and antibodies (classical pathway) are parallel pathways for initiating the opsonization pathway of complement, which is an important effector in tagging and removal of foreign bodies such as invading pathogens. Unlike vaccines, which require days to train the adaptive immune response and days to produce antibodies, the innate immune system is always active. As part of our innate immune system, MBLs can respond to pathogens immediately, enabling therapeutic and preventative applications.

4.0 Coronavirus Variants and their Similarities

COVID-19 is caused by a coronavirus known as SARS-CoV-2, which is genetically very similar to Severe Acute Respiratory Syndrome CoronaVirus (SARS-CoV-1). Coronaviruses are enveloped viruses. SARS-CoV-2 proteins are highly homologous (95%-100%) to proteins of the SARS-CoV-1 virus. For example, the SARS-CoV-2 spike (S) protein has roughly 75% amino acid identity with SARS-CoV-1 with the spike receptor binding domain approximately 73% conserved (retained, unchanged).

The SARS-CoV-2 S protein is the main target for antibodies during infection and a focus for vaccine design because it is virtually the only antigen present on the virus surface. The SARS-CoV-2 S protein is extensively glycosylated with 22 N-linked glycan sequons per protomer. SARS-CoV-1 S protein possesses 23 N-linked glycosylation sequons per protomer, with 20 of the 22 SARS-CoV-2 S glycosylation sequons conserved from SARS-CoV-1.

Furthermore, the 22 SARS-CoV-2 S glycosylation sequons are highly conserved amongst predominant variants of SARS-CoV-2. Site-specific glycosylation analysis indicates that mutations in variants do not affect the S glycans. This may be due to the critical role some S glycans have in the opening of the spikes as part of an infection process. Currently, all major SARS-CoV-2 variants have these same conserved sites. Because these sites are conserved, they present an attractive target as a potential pan-variant therapeutic intervention, such as compositions developed and described herein, against SARS-CoV-2.

5.0 Mannose Binding Lectins and Coronaviruses

MBLs exhibit significant activity against enveloped viruses, such as SARS-CoV-1, as their glycosylated spikes allow MBLs to interfere at two points of the viral life cycle: viral entry and viral shedding.

Site-specific glycosylation analysis suggests that the glycan shield of coronaviruses exhibits numerous vulnerabilities. SARS-CoV-1, for example, has a broad spectrum of underprocessed oligomannose-type glycans that enable innate immune system recognition of coronaviruses by MBLs. Thus, targeting these sites is aiming for a critical weakness that the SARS-CoV-1 needs to be infectious.

Prior studies have also indicated that MBL deficient individuals are more susceptible to coronaviruses, such as SARS-CoV-1. Innate immune systems with insufficient levels of coronavirus effective MBLs are more easily overwhelmed, resulting in infection, symptom expression, and disease. By contrast, hosts capable of maintaining sufficient levels of coronavirus effective MBL could maintain a sufficient innate immune system response until the adaptive immune system responds to produce sufficient antibodies. As MBLs and the innate immune defense are the first line host defense against pathogens, lectin levels may contribute to both susceptibility and outcome (e.g., whether an infected host exhibits symptoms or remains asymptomatic). The innate immune system may also be capable of clearing an infection on its own as at least one SARS-CoV-2 patient recovered from COVID-19 with no detectable IgG.

6.0 Plant Lectins

Similar to our innate immune system, a plant's innate immune system uses lectins to defend against pathogens. A number of plant lectins, especially plant MBLs, exhibit properties of varying efficacy against certain pathogens. However, it is noted that, as plants each have their own innate immune system producing their own type of lectin, there are many possible plant lectins which make it difficult to identify ones that may be effective against a particular pathogen. As discussed herein, the various structures, conformations, and variations of a particular lectin defines its binding affinity to a specific pathogen. TABLE 1 lists some example structures for example lectins.

TABLE 1

| Lectin(s) | Example Structure(s) |
|---|---|
| Urtica dioica agglutinin | Monomer |
| | Homodimer |
| Nicotiana tabacum agglutinin | Homodimer |
| Allium ascalonicum agglutinin | Dimer |
| Allium sativum agglutinin | Dimer |
| (ASA I/II/III Group) | Tetramer |
| Allium porrum agglutinin | Homotrimer |
| BanLec | Dimer |
| | Tetramer |
| Ananas comosus agglutinin | Homodimer |
| | Tetramer |
| Allium cepa agglutinin | Tetramer |
| Galanthus nivalis agglutinin | Tetramer |

As a result of these many confounding factors, various plant lectins exhibit anti-coronavirus properties of varying efficacy (ranging from nil to highly effective) which makes it difficult to identify ones that may be sufficiently effective and safe for use.

Both in silico and in vitro experiments subsequently performed by Inventor confirm that certain plant lectins, including Allium porrum agglutinin, are capable of identifying both SARS-CoV-1 and SARS-CoV-2 as a pathogen.

Inventor has invented a composition including one or more plant lectins as a viable approach to minimizing at least a COVID-19 infection risk, as plant lectins are effective, fast-acting, affordable, and accessible, and complement vaccines. At least one of the one or more plant lectins in the composition binds to the SARS-CoV-2 virus and activates our complement system which thereby targets the innate immune system against these pathogens.

Where reference is made to a specific plant lectin, it should be appreciated that there may be natural or non-natural variations in the genetic and/or protein sequence for a plant lectin such that minor variations in the genetic sequence and/or protein sequence would yield a lectin of sufficient similarity in both form and function to be referred to and within the scope of the named plant lectins. A lectin thus derived from a plant lectin with minor mutations and/or variations with similar form and function is considered within the scope of the named plant lectin.

7.0 Artificial Intelligence and Machine Learning

As discussed herein, lectins are a very powerful molecular pattern recognition tool activating the immune system. Lectins can be particularly useful for targeting glycosylated pathogens that vaccines and antibodies are not well suited for.

Artificial intelligence and machine learning techniques may be employed to identify and determine (predict, generate, identify) which lectins can recognize certain PAMPs to prioritize further research into which lectins are the most effective at activating the immune system in order to identify sources of those lectins, and to identify and determine (predict, generate, identify) genetically modified lectins that more effectively targets particular pathogens and associated PAMPs.

Artificial intelligence and machine learning techniques include any one or more of: supervised learning (e.g., using gradient boosting trees, using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), glowworm swarms, and/or any suitable artificial intelligence/machine learning techniques.

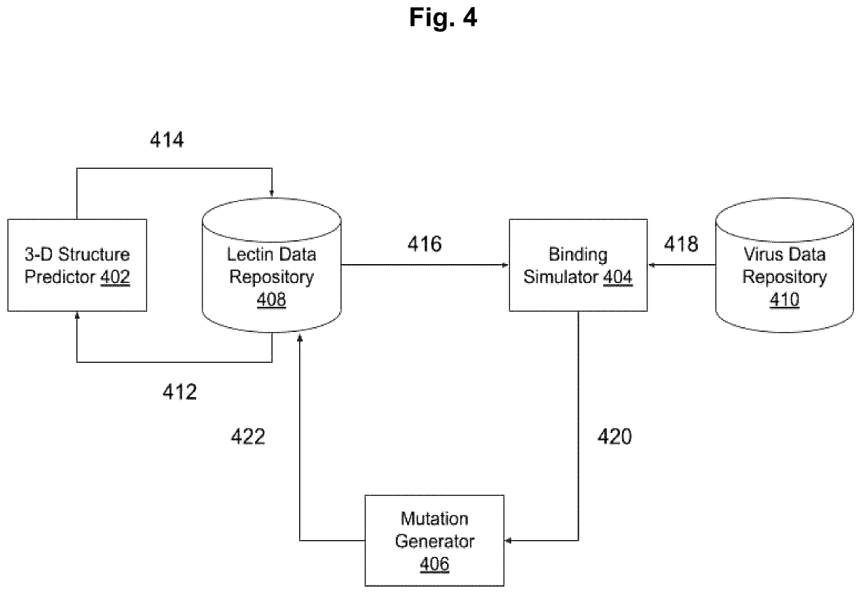

FIG. 4 illustrates an example data flow 400 that may represent an algorithm to form the basis of programming a computer to implement mutation generation using feedback data in accordance with some embodiments. The data flow 400 includes two loops in which data flow from and to a mutation generator 406. The first loop is shown at the left of FIGS. 4 (408, 412, 402, and 414), and a second loop is shown at the right of FIGS. 4 (408, 416, 404, 420, and 406).

Referring to the first loop, at Step 412, a lectin is selected from a lectin data repository 408 using, for example, a computing device (not illustrated). In an embodiment, the lectin may be selected from a protein database of the repository 408 in accordance with a priority list based on the structure of the lectin and target receptor, a pre-mutation binding score, or manually via a command line, configuration, or graphical user interface. A protein database may include a publicly accessible database, for example, RCSB PDB (Research Collaboratory for Structural Bioinformatics Protein Data Bank), Uniprot (Universal Protein Resource), or AlphaFold Protein Structure Database, and/or a private database. In an embodiment, each protein in the lectin data repository 408 is associated with a notation and/or file format that can be understood by a 3-D structure predictor 402, which may include, for example, an AlphaFold-based model that uses AI to predict protein structures based on their amino acid sequence(s). As an example, a FASTA-type file format may be used to provide protein sequence input(s) to the 3-D structure predictor 402.

At Step 414, the structure predictor 402 predicts (identifies, generates, determines) at least one 3D structure for a lectin from the lectin data repository. The at least one predicted 3D structure of the lectin is associated with the lectin in the lectin data repository 408. In an embodiment, the at least one predicted 3D structure of the lectin may be stored in a structures database of the lectin data repository 408. As an example, a PDB-type file format may be used to store a predicted lectin structure. Multiple predicted 3D structures may be associated with a lectin to represent different conformations when, for example, there is flexibility in the structure allowing for a range of positions for carbohydrate recognition domains. In an embodiment, each 3D structure in the structures database is associated with a representation that can be understood by a binding simulator 404, which may include, for example, a LightDock-based simulator.

In an embodiment, Steps 412 and 414 are repeated for every lectin with an unknown structure in the repository 408 to obtain a corresponding predicted 3D structure. Lectins in the repository 408 includes natural lectins (e.g., found in plants) and modified lectins (e.g., generated by the mutation generator 406). The repository 408 may be continuously updated with new lectins and structures.

Referring to the second loop, at Step 416, a lectin for which there is a known or predicted 3-D structure, is selected from the lectin data repository 408 using, for example, a computing device (not illustrated). In an embodiment, the known or predicted 3D structure may be selected from the structures database of the lectin data repository 408 via a graphical user interface.

At Step 418, one or more virus(es) is/are selected from a virus data repository 410 using, for example, a computing device (not illustrated). In an embodiment, each virus in the virus data repository 410 may be associated with one or more binding (e.g., glycosylation) sites, each having a representation that can be understood by the binding simulator 404.

At Step 420, one or more scores are generated by the binding simulator 404. A score generated by the binding simulator 404 represents how well a lectin is predicted to bind to the virus (e.g., binding affinity). In an embodiment, the binding simulator 404 may be based on one more scoring functions, such as TOBI potentials, DFIRE/DFIRE2, pyDock, Mj3h, and SwarmDock. These scoring functions estimate or approximate binding energies between a receptor and ligand to identify potential low energy binding conformations suggesting a possible binding and conformation between the receptor and ligand.

In an embodiment, the binding simulator 404 may also predict (identify, generate, determine) a set of one or more lectins which may be effective against a set of one or more viruses. For example, depending on the input to the binding simulator 404, the binding simulator 404 may determine binding affinities between an input lectin and multiple viruses from the virus data repository 410, or binding affinities between an input virus and multiple lectins from lectin data repository 408.

At Step 422, genetically modified lectin candidates are generated by the mutation generator 406 which includes at least one AI/ML model. The mutation generator 406, with the scoring functions of the binding simulator 404, digitally simulates an evolutionary process optimizing one or more lectins binding to one or more pathogens.

The mutation generator 406 introduces single-point mutations, which are the most frequent type of mutation, where a single nucleotide is changed (e.g., substitution, deletion or insertion) in, for example, a DNA or RNA sequence resulting in a different protein or amino acid sequence. The processing (second) loop includes mutation generator 406 for iterating over single-point mutations optimizing binding per the scoring function to digitally “evolve” lectin(s) optimized for binding to one or more pathogens. This iteration process may be optimized by incorporating known and/or predicted binding information from lectin data repository 408 and/or virus data repository 410, and may include a pruning process, such as considering only the top n candidates, to enhance computational efficiency by avoiding consideration of losers in this digital evolutionary process. The value for n may be fixed (e.g., a constant number between 100 to 100,000), based on the input protein or nucleotide sequence length(s), and/or based on the binding scores from binding simulator 404.

The artificial intelligence and machine learning techniques disclosed may be used to identify, based on predicted lectin binding affinity to PAMPs on pathogens, one or more plant lectins and derivatives that may be effective for mitigating viral risks (e.g., COVID-19, HIV, influenza) and/or for improving overall public health. Plant lectins are cheap, replenishable, readily available, and may be manufactured with recombinant technologies. An example process of manufacturing lectins is described in U.S. provisional application 63/446,407, filed Feb. 17, 2023, entire contents of which are hereby incorporated herein by reference for all purposes as if fully set forth herein.

An optimized lectin may be stored in the lectin data repository 408 for subsequent processing. For example, an optimized lectin may be used by the binding simulator 404 to identify other pathogens it may have functional binding affinity. For another example, the optimized lectin may be further optimized by the mutation generator 406 for efficacy against one or more pathogens.

8.0 Flavonoids

Flavonoids are a class of polyphenol compounds commonly found in plants with a wide range of functions and are commonly consumed by humans. Effects of flavonoids on the human immune system are recently being studied. Some flavonoids have demonstrated medicinal benefits including anticancer, antioxidant, anti-inflammatory, and antiviral properties where recent research has identified some flavonoids capable of inhibiting viral reproduction. For example, some flavonoids bind to the main protease of SARS coronaviruses, which is required for cell proliferation and maturation, thereby inhibiting a pathogens' reproduction.

Flavonols are a class of flavonoids which have a 3-hydroxyflavone backbone. Flavonols are present in a wide variety of plants, particularly fruits and vegetables. Several flavonols have demonstrated immunomodulating activity. Example flavonols that are bioactive against SARS-CoV-2 include kaempferol and quercetin.

Flavanols are a class of flavonoids which have a 2-phenyl-3,4-dihydro-2H-chromen-3-ol skeleton, and include catechins such as epigallocatechin gallate (EGCG). Flavanols are present in a wide variety of plants, particularly fruits and teas. EGCG is an example flavanol that is bioactive against SARS-CoV-2 by, for example, inhibiting the activity of the 3CL-protease (3CL^{pro}) or main protease (M^{pro}) of the coronavirus.

Although specific flavonols (e.g., kaempferol and quercetin) and flavanols (e.g., EGCG) are mentioned, their related derivatives and forms (e.g., kaempferitrin, rutin, and spiraeoside) are considered within scope of the general form mentioned.

9.0 Calcium

Calcium is a mineral that all living organisms, including humans, need. Calcium plays many key roles in the human body such as contributing to bone development, muscle contraction, and blood clotting. Furthermore, the presence of calcium helps MBLs, a C-type lectin, bind to conserved glycans on SARS coronaviruses. The calcium component may be in one of many forms, such as calcium phosphate, calcium citrate, calcium carbonate, calcium chloride, calcium gluconate, and/or as present in saline (e.g. hypotonic, isotonic, or hypertonic saline).

10.0 Composition for Pattern Recognition Based Targeting and Activating an Innate Immune Response

Inventor has developed a composition, as illustrated in FIG. 3, that enables an innate immune response against SARS coronaviruses; a complement to vaccines training our adaptive immune system. The composition is effective, fast-acting, affordable, and accessible. The composition may be administered to a healthy person to prevent, for example, a SARS-CoV-2 infection or a person infected with the SARS-CoV-2 virus and COVID-19 for treatment. The composition is configured for broad spectrum prototypic pattern recognition based targeting of the SARS coronavirus (S) protein and activating an innate immune response.

The composition includes a lectin component. The lectin component is generally safe for consumption and is not known to frequently cause autoimmune reactions with less than 10% of the population having an allergic reaction. The lectin component comprises at least one lectin capable of binding to at least the SARS coronavirus that, upon binding, acts as an opsonin. A lectin in the lectin component may be a natural lectin or a manufactured lectin. A natural lectin may be extracted or isolated from, for example, Allium porrum, Urtica dioica, or Nicotiana tabacum. Examples of other natural lectins are shown in Table 1. A manufactured lectin may be synthesized or derived from a plant, such as Allium porrum, Urtica dioica, or Nicotiana tabacum. An example process of manufacturing lectins is described in U.S. provisional application 63/446,407, filed Feb. 17, 2023, entire contents of which are hereby incorporated herein by reference for all purposes as if fully set forth herein. A lectin may comprise one or more subunits resulting in one of, for example, a monomer, dimer, trimer, or tetramer structure. In each of the multimer forms, a lectin may consist of identical subunits (denoted with a homo-prefix) or different subunits (denoted with a hetero- prefix). A homotrimeric lectin, for example, refers to a lectin consisting of 3 identical subunits. Some lectins may be capable of multiple forms (e.g., both as a homo-dimer and hetero-tetramer) where the lectin's structure determines its binding affinity and selectivity.

In an embodiment, the composition may also include a pharmaceutical component. The pharmaceutical component may be a pharmaceutically-acceptable carrier (e.g., saline) and/or excipient suitable for administering and delivering the composition. Pharmaceutically-acceptable examples include saline, gelatin, and enteric coatings.

In an embodiment, the composition may also include a flavonoid component. The flavonoid component functions as a reproduction inhibitor for at least the SARS coronaviruses. The flavonoid component comprises at least one flavonoid, such as a flavonol (e.g., kaempferol, quercetin, etc.) or flavanol (e.g., EGCG), capable of binding to the main protease of the SARS-CoV-2 virus and inhibiting its function for viral replication.

In an embodiment, the composition may also include an optional supportive component. The supportive component facilitates the lectin component binding to pathogens, including the SARS coronavirus. Calcium is an example supportive component which may be provided in one of many forms including, for example, calcium phosphate, calcium citrate, calcium carbonate, calcium chloride, calcium gluconate, and/or in a pharmaceutical component.

Ingredients or components of the composition may be in different forms including but not limited to powder, syrup, pulp, concentrate, extract form, isolate, and liquid. Various embodiments of the composition may be administered orally, intravenously, nasally, or inhaled to enable an innate immune response against SARS coronaviruses, such as SARS-CoV-2.

10.1 Concentration Determinations

As MBL is a key innate immune system pathogen recognizer, it is worthwhile to determine an appropriate amount of MBL for mitigating COVID-19 risk. Previous research has found blood MBL concentrations vary widely in people from undetectable (e.g., 0 μg/mL) to as high as 10 μg/mL. The maximum amount of MBL in an average adult is thus 50,000 μg, based on an average amount of blood in an adult is 5,000 mL. Blood accounts for approximately 7-8% of an adult's body weight, 8-9% of a child's body weight, and approximately 9-10% of an infant's body weight. An average female weighing 165 lbs/74.8 kg has about 4.3 L of blood whereas an average male weighing 200 lbs/90.7 kg has about 5.7 L of blood. Infants have approximately 75-80 mL of blood per kg and children have about 70-75 mL of blood per kg. Estimating blood volume allows scaling the composition of the present invention to an appropriate dose.

Despite the wide variance in MBL concentrations, a prior study found that the median serum level of MBL in SARS-CoV-1 patients was significantly lower (0.733 μg/mL) than that in control (healthy) subjects (1.369 μg/mL); an average MBL concentration difference of 0.636 μg/mL. For the average adult, this translates to a difference of 3,180 μg, which represents an estimated amount for the average adult MBL deficiency between healthy control and SARS-CoV-1 patients. The prior study also found that the maximum difference (delta) of MBL level between control subjects (2.598 μg/mL) and the SARS-CoV-1 patients (0.263 μg/mL) was 2.335 μg/mL. Thus, an amount of 11,675 μg of MBL would overcome the maximum delta in MBL levels for an average adult. Overcoming the MBL deficiency (0.636 μg/mL) provides benefits against SARS coronaviruses, subject to limits on the maximum MBL levels (10 μg/mL) and maximum MBL level difference (delta=2.335 μg/mL) identified limiting the bioavailable amount of MBL in our composition to approximately 11,675 μg, as measured in blood serum or whole blood. Benefits are dependent at least on quantity administered and variations in bioavailability, including as a result of different delivery methods.

Table 2 shows effective concentrations of example plant lectins against SARS coronaviruses.

TABLE 2

| | | | μg |
|---|---|---|---|
| | | EC₅₀ | per average |
| Lectin | Plant Source | (μg/mL) | adult for EC₅₀ |
| Allium Porrum | Allium Porrum | 0.45 | 2,250 |
| Agglutinin (APA) | | | |
| Urtica dioica | Urtica dioica | 1.3 | 6,500 |
| Agglutinin (UDA) | | | |
| Nicotiana tabacum | Nicotiana Tabicum | 1.7 | 8,500 |
| agglutinin (Nictaba) | | | |
| Mistletoe Lectin II | Viscum album | 0.015 | 75 |

APA is the most effective lectin against SARS-CoV-1 with an EC₅₀ of 0.45 μg/ml, followed by UDA and Nictaba with an BCH, of 1.3 μg/ml and EC₅₀ of 1.7 μg/ml, respectively. For the average adult, this translates to 2,250 μg of APA, 6,500 μg of UDA, and 8,500 μg of Nictaba to reach the EC₅₀ level against SARS coronaviruses.

Preferably, the amount of lectin in the composition, both for a single dose and maximum blood concentration, does not significantly exceed 2.335 μg/mL or 11,675 μg for the average adult. In an embodiment directed to therapeutic applications, an amount of lectin in the composition for a single initial therapeutic dose for an average adult is between a first amount of lectin necessary to reach the EC₅₀ level (e.g., 0.45 μg/mL or 2,250 lag) and a second amount overcoming the maximum delta (e.g., 2.335 μg/mL or 11,675 μg). In an embodiment directed to preventative applications, an amount of lectin in the composition for a single initial therapeutic dose for an average adult is between a first amount that is at least one half of 1/7th the amount of lectin (160 μg) necessary to reach the EC₅₀ level and a second amount of approximately 3 times the EC₅₀ level (e.g., 1.35 μg/mL or 6,750 lag). It is important that, at any time, the amount of lectin in the body remains within a safe level not exceeding the maximum concentration level of 10 μg/mL and preferably not significantly exceeding the concentration level of 2.335 μg/mL discussed above (11,675 μg for the average adult). Further studies may determine a more concrete safety level.

MBL has a half life of between 3-7 days. A longer 7-day half life means 50% of the MBL administered remains after about 7 days. A shorter 3-day half life means 50% of the MBL administered remains after about 3 days. If 3 μg/mL worth of MBL is administered in a subject, then in 3-7 days, there is 1.5 ug/mL worth of MBL left in the subject. To maintain a sufficient amount of MBL in our bloodstream, subsequent doses are required and the amount of MBL in our bloodstream needs to account for the half life and how often it's administered.

Different doses of the composition to account for bioavailability and/or for preventative and therapeutic purposes are contemplated. While therapeutic applications require a higher amount of lectin to be effective against a higher viral load, preventive applications allow for a lower amount of lectin in the composition, where a single dose may include, for example, between 160 lag and 6,750 μg of APA, corresponding to one half of 1/7th the EC₅₀ level up to triple the EC₅₀ level. These doses may be administered daily, weekly, or as necessary.

An example dosage schedule to recover from a SARS infection may be a single higher dose of 320 μg-1,607 μg of MBL for up to each of the first three days, followed by a single dose of 35 μg-320 μg of MBL for each of the next four days (subject to the limitations described above). Other dosage schedules are contemplated, depending on intended purpose and severity of infection. For example, Table 3 shows example dosage schedules for therapeutic and preventative applications for an average adult, to illustrate various target ranges and applications of APA in blood serum.

TABLE 3

| Day | Therapeutic (mg of APA) | | | | | Prevention (mg of APA) | | | | |
|---|---|---|---|---|---|---|---|---|---|---|
| 0 | 7.5 | 10.0 | 3.0 | 10.0 | 5.0 | 0.25 | 0.5 | 1.0 | 3.5 | 5.0 |
| 1 | 0.5 | 1.0 | 3.0 | 0.0 | 0.0 | 0.25 | 0.5 | 1.0 | 0.0 | 0.0 |
| 2 | 0.5 | 1.0 | 3.0 | 0.0 | 0.0 | 0.25 | 0.5 | 1.0 | 0.0 | 0.0 |
| 3 | 0.5 | 1.0 | 0.5 | 0.0 | 0.0 | 0.25 | 0.5 | 1.0 | 0.0 | 0.0 |
| 4 | 0.5 | 1.0 | 0.5 | 0.0 | 0.0 | 0.25 | 0.5 | 1.0 | 0.0 | 0.0 |
| 5 | 0.5 | 1.0 | 0.5 | 0.0 | 0.0 | 0.25 | 0.5 | 1.0 | 0.0 | 0.0 |
| 6 | 0.5 | 1.0 | 0.5 | 0.0 | 0.0 | 0.25 | 0.5 | 1.0 | 0.0 | 0.0 |
| 7 | 0.5 | 1.0 | 0.5 | 5.0 | 5.0 | 0.25 | 0.5 | 1.0 | 3.5 | 5.0 |
| 8 | 0.5 | 1.0 | 0.5 | 0.0 | 0.0 | 0.25 | 0.5 | 1.0 | 0.0 | 0.0 |
| 9 | 0.5 | 1.0 | 0.5 | 0.0 | 0.0 | 0.25 | 0.5 | 1.0 | 0.0 | 0.0 |
| 10 | 0.5 | 1.0 | 0.5 | 0.0 | 0.0 | 0.25 | 0.5 | 1.0 | 0.0 | 0.0 |
| 11 | 0.5 | 1.0 | 0.5 | 0.0 | 0.0 | 0.25 | 0.5 | 1.0 | 0.0 | 0.0 |
| 12 | 0.5 | 1.0 | 0.5 | 0.0 | 0.0 | 0.25 | 0.5 | 1.0 | 0.0 | 0.0 |
| 13 | 0.5 | 1.0 | 0.5 | 0.0 | 0.0 | 0.25 | 0.5 | 1.0 | 0.0 | 0.0 |
| 14 | 0.5 | 1.0 | 0.5 | 0.0 | 0.0 | 0.25 | 0.5 | 1.0 | 0.0 | 0.0 |

In an embodiment, the composition may also include calcium. A purpose for including calcium in the composition is to support and facilitate MBL binding. Factors to consider when determining the amount of calcium to include in the composition include (but are not limited to) effects on bioavailability, efficacy at improving lectin binding, avoiding oversaturation, safety (where, for example, excess calcium may exacerbate some issues including, for example, kidney stones), and compatibility with delivery methodology.

Calcium is present with lectins in many plants; however, the ratio of calcium to lectins varies widely including across different species within the same genus. For example, Allium sativum has a ratio of approximately 0.9:1 Ca to MBL ratio while the ratio of calcium to MBL in Allium porrum is 59:1. Even within the Allium cepa species, Allium cepa var. cepa has an approximate calcium to lectin ratio of 23:1, whereas Allium cepa var aggregatum has an approximate calcium to lectin ratio of 37:1. The highest of these ratios translates to approximately 132.75 mg of calcium for supporting 2,250 μg of MBL. The lowest of these ratios translates to approximately 2 mg of calcium to support MBL function when 2,250 μg of APA is present. In an embodiment, the lectin component may be administered in a hypotonic, isotonic, or hypertonic saline solution containing calcium; and, if desired, additional calcium may be supplemented as necessary.

In an embodiment, an amount of calcium in the composition for a single dose is based on the ratio of calcium to lectin in an amaryllidaceae family plant (e.g., Allium cepa, allium porrum, allium sativum, or Allium schoenoprasum in the genus Allium of the family amaryllidaceae) and/or on the total amount of the lectin in the composition.

Dietary calcium absorption is up to −50% but is often less. Providing up to 60:1 of calcium to MBL is contemplated in addition to adjustments for absorption and bioavailability. Similar calculations could be performed to determine respective amounts of calcium based on different calcium to MBL ratios. As calcium is often readily available, additional calcium may also be administered with the composition depending on the recipients' need.

In an embodiment, the composition may also include at least one flavonoid, e.g., a flavonol or flavanol. A purpose of including one or more flavonoids in the composition is to inhibit viral reproduction. While flavonoids are present with lectins in many plants, the ratio of a particular flavonoid to lectins varies widely even across different species within the same genus. As one example, Allium sativum has very low ratios of approximately 3:2000 kaempferol to MBL and a ratio of 17:2000 quercetin to lectin. As another example, Allium cepa var. cepa has higher ratios of approximately 3:5 kaempferol to MBL and a ratio of approximately 20:1 quercetin to MBL. In between, Allium porrum has a more moderate ratio of approximately 2.7:1 kaempferol to MBL and a ratio of approximately 0.9:1 of quercetin to MBL. These moderate ratios translate to approximately 6.08 mg of kaempferol and approximately 2.03 mg of quercetin when 2,250 μg of APA is present. Similar calculations may be performed to determine a lower bound estimate using Allium sativum and higher bound estimate with Allium cepa var. cepa for an amount of flavonol in the composition.

In an embodiment, an amount of a flavonol in the composition for a single dose is based on the ratio of that flavonol to lectin in the amaryllidaceae family or Allium genus (e.g., Allium porrum) and/or based on the amount of the lectin component in the composition. For example, an amount of kaempferol may range from approximately 3.03 mg to approximately 12.15 mg when 2,250 μg of APA is present, which can translate to approximately 0.43 mg to approximately 4.07 mg of kaempferol in a single daily dose. For another example, an amount of quercetin may range from approximately 1.01 mg to approximately 4.05 mg when 2,250 μg of APA is present, which can translate to approximately 0.14 mg to approximately 0.57 mg of quercetin in a single daily dose.

Other methods of determining an amount of a flavonoid are contemplated. For example, an amount of a flavonol in the composition for a single dose can be determined based on the EC₅₀ level of the flavonol. Using quercetin as an illustration, an amount of quercetin may range from the amount of quercetin necessary to reach the EC₅₀ level (60 Kg/ml or 300 mg) and a level not to exceed 5 times that amount (300 μg/ml or 1,500 mg) for an average adult. Using kaempferol as another illustration, an amount of kaempferol may range from the amount of kaempferol necessary to reach the EC₅₀ level (10 μg/ml or 50 mg) and a level not to exceed 5 times that amount (50 μg/ml or 250 mg) for an average adult.

In an embodiment, an amount of a flavonoid in the composition for a single dose is based on the effectiveness of that flavonoid against the target pathogen(s) and the flavonoid's safety profile to yield a desired range of 10% to 20× the IC50 level. EGCG (˜458.4 g/mol) has in vitro IC50 values ranging from approximately 0.8471.1M to 16.5 μM against SARS-CoV-2 main protease. Thus, an example daily amount of EGCG at the IC50 level may range from approximately 1.9 mg to approximately 37.8 mg for an average adult where the dosage may be computed in accordance with well-known approaches and as exemplified above to target a desired range of 10% to 20× the IC50 levels (e.g., between approximately 0.19 mg and 756 mg per day) preferably remaining within levels considered safe (e.g., less than or equal to approximately 500 mg per day).

Quercetin and kaempferol both have a half life of less than 28 hours. Thus, to maintain sufficient amounts of one or more flavonols to effectively inhibit viral reproduction, quercetin, kaempferol, or both may need to be administered daily at a suitable dosage range to account for the half life. Similarly, EGCG has a half life of approximately 4 to 5 hours. In an embodiment, the suitable dosage range may be computed in accordance with well known approaches and as exemplified with the lectins above.

Table 4 shows approximate IC50 concentrations of example flavonoids against SARS coronaviruses with approximate IC50 values for the flavonoid component.

TABLE 4

| Flavonoid | IC₅₀ (μg/mL) |
|---|---|
| Kaempferol | 62.50-125.00 |
| Quercetin | 7.19-60 |
| EGCG | 0.389-7.56 |
| Luteolin | 5.72 |
| Cinnamtannin B1 | 28.4 |
| Hesperetin | 2.5 |
| Myricetin | 0.86 |
| Pristimerin | 2.55 |

It is noted that the amounts/ranges provided herein are exemplary and should not be construed to limit the disclosed invention. Similar calculations could be performed to determine amounts of other plant lectins, amounts of calcium, and amounts of one or more flavonols based on the other plant lectins in the composition. While target concentrations in blood are disclosed, the amount of each ingredient/component of the composition may be adjusted according to bioavailability of the ingredient and/or to how the composition is administered (e.g., delivery method).

10.2 Example Composition 1

A first example composition comprises a lectin component. This example composition may be administered orally, intravenously, nasally, or inhaled to provide a line of defense against SARS coronaviruses, such as SARS-CoV-2 dependent on the amount of blood in an individual taking the first example composition.

The lectin component includes one or more lectins. In an embodiment, for a single therapeutic dose, the amount of a lectin in the first example composition ranges between an amount of lectin necessary to reach half of 1/7th the amount of lectin necessary to reach the EC₅₀ level (e.g., (½)*( 1/7)*0.45 μg/mL or 160 μg) and a second amount overcoming the maximum delta (e.g., 2.335 μg/mL or 11,675 μg). In an embodiment, for a single preventative dose, the amount of a lectin in the first example composition is at least half of 1/7th the amount of lectin necessary to reach the EC₅₀ level of lectin but no more than an amount overcoming the maximum delta (e.g., 2.335 μg/mL or 11,675 μg).

In an embodiment, the lectin component includes Allium porrum agglutinin. In an embodiment, the lectin component also includes, in addition to Allium porrum agglutinin, at least one of Urtica dioica agglutinin and Nicotiana tabacum agglutinin. In an embodiment, the lectin component also includes, in addition to Allium porrum agglutinin, a lectin having a dimeric structure and/or a lectin having a tetrameric structure. In an embodiment, the lectin component also includes at least two of: a lectin having a dimeric structure, a lectin having a trimeric structure, and a lectin having a tetrameric structure. An example lectin with a dimeric structure is Allium sativum agglutinin. An example lectin with a trimeric structure is Allium porrum agglutinin. An example lectin having a tetrameric structure is Allium cepa agglutinin

10.3 Example Composition 2

A second example composition comprises a lectin component and a flavonoid component. This example composition may be administered orally, intravenously, nasally, or inhaled to provide a line of defense against SARS coronaviruses, such as SARS-CoV-2.

The amount of the lectin component in the second example composition may be the same as that described above for the first example composition and is not repeated in this section for purposes of clarity and brevity.

The flavonoid component may include one or more flavonols. In an embodiment, for a single dose, the amount of the flavonoid component in the second example comprises an amount ranging between approximately 0.24 μg (or 0.048 μg/mL) to approximately 31.52 mg (or 6.30 μg/mL) of kaempferol, an amount ranging between approximately 1.36 μg (or 0.272 ng/mL) to approximately 233.5 mg (46.7 μg/mL) of quercetin, or both.

In an embodiment, for a single dose, the amount of a first flavonol in the second example composition includes the amount of the first flavonol necessary to reach the EC50 level of the first flavonol but no more than five times that amount, the amount of a second flavonol in the second example composition includes the amount of the second flavonol necessary to reach the EC50 level of the first flavonol but no more than five times that amount, or both, wherein the first flavonol is different from the second flavonol. Using quercetin as an illustration, an amount of quercetin may range from the amount of quercetin necessary to reach the EC50 level (60 μg/ml or 300 mg) and a level not to exceed 5 times that amount (300 μg/ml or 1,500 mg) for an average adult. Using kaempferol as another illustration, an amount of kaempferol may range from the amount of kaempferol necessary to reach the EC50 level (10 Kg/ml or 50 mg) and a level not to exceed 5 times that amount (50 μg/ml or 250 mg) for an average adult.

The flavonoid component may include, instead of or in addition to the one or more flavonols, one or more flavanols. In an embodiment, for a single dose, the amount of the flavonoid component in the second example comprises an amount ranging between approximately 0.19 mg (or 0.038 μg/mL) to approximately 189 mg (or 37.8 μg/mL) of EGCG with calculations similar to those above for the flavonol.

In an embodiment, for a single dose, the amount of the flavonoid component in the second example composition is dependent on the flavonoid(s) included, the ratio of flavonoid to lectin in a plant such as one from the amaryllidaceae family (e.g., from the Allium genus), and also on the amount of the lectin component in the second example composition.

10.4 Example Composition 3

A third example composition comprises a lectin component and a supportive component. This example composition may be administered orally, intravenously, nasally, or inhaled to provide a line of defense against SARS coronaviruses, such as SARS-CoV-2.

The amount of the lectin component in the third example composition may be the same as that described above for the first example composition and is not repeated in this section for purposes of clarity and brevity. The supportive component includes calcium.

In an embodiment, for a single dose, the amount of the supportive component in the third example composition is based on at least 0.9 Ca to lectin ratio (e.g., 2 mg Ca for 2,250 μs APA) but no more than a typical daily dose of calcium (e.g., 625 mg).

In an embodiment, the supportive component may include saline (e.g. hypotonic, isotonic, or hypertonic saline).

In an embodiment, the amount of the supportive component of the third example composition is dependent on the ratio of calcium to lectin in a plant such as one from the amaryllidaceae family (e.g., from the Allium genus) and also on the amount of the lectin component in the second example composition.

10.5 Example Composition 4

A fourth example composition comprises a lectin component, a flavonoid component, and a supportive component. This example composition may be administered orally, intravenously, nasally, or inhaled to provide a line of defense against SARS coronaviruses, such as SARS-CoV-2.

The amount of the lectin component in the fourth example composition may be the same as that described above for the first example composition and is not repeated in this section for purposes of clarity and brevity. The amount of the flavonoid component in the fourth example composition may be the same as that described above for the second example composition and is not repeated in this section for purposes of clarity and brevity. The amount of the supportive component in the fourth example composition may be the same as that described above for the third example composition and is not repeated in this section for purposes of clarity and brevity.

11.0 Hardware Overview

According to one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as at least one application-specific integrated circuit (ASIC) or field programmable gate array (FPGA) that is persistently programmed to perform the techniques or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

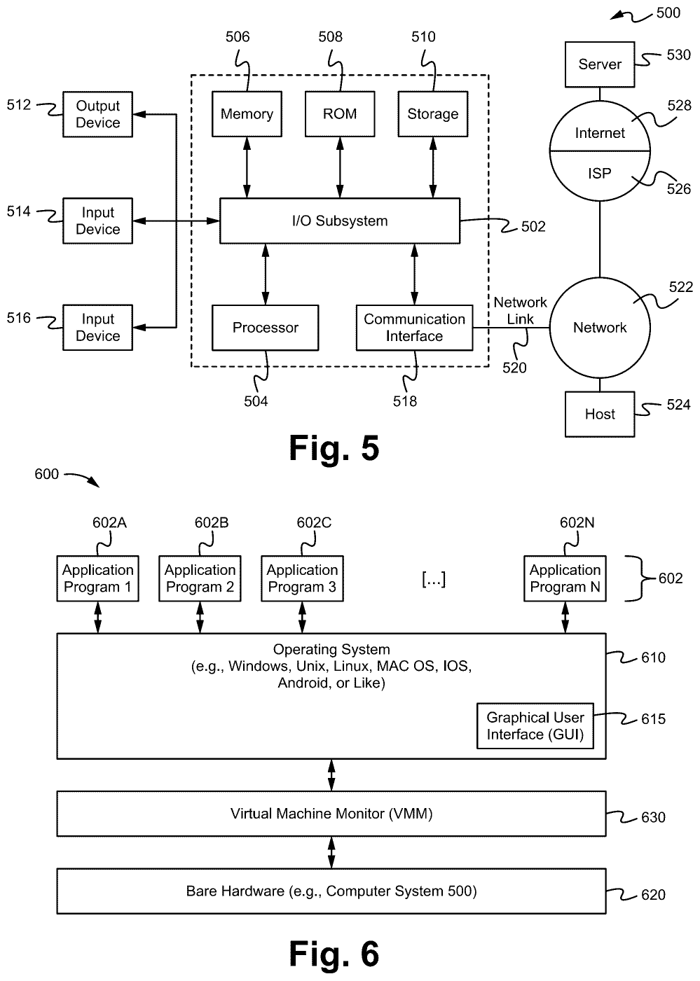

FIG. 5 is a block diagram that illustrates an example computer system with which an embodiment may be implemented. In the example of FIG. 5, a computer system 500 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 500 includes an input/output (I/O) subsystem 502 which may include a bus and/or other communication mechanism(s) for communicating information and/or instructions between the components of the computer system 500 over electronic signal paths. The I/O subsystem 502 may include an I/O controller, a memory controller and at least one I/O port. The electronic signal paths are represented schematically in the drawings, for example as lines, unidirectional arrows, or bidirectional arrows.

At least one hardware processor 504 is coupled to I/O subsystem 502 for processing information and instructions. Hardware processor 504 may include, for example, a general-purpose microprocessor or microcontroller and/or a special-purpose microprocessor such as an embedded system or a graphics processing unit (GPU) or a digital signal processor or ARM processor. Processor 504 may comprise an integrated arithmetic logic unit (ALU) or may be coupled to a separate ALU.

Computer system 500 includes one or more units of memory 506, such as a main memory, which is coupled to I/O subsystem 502 for electronically digitally storing data and instructions to be executed by processor 504. Memory 506 may include volatile memory such as various forms of random-access memory (RAM) or other dynamic storage device. Memory 506 also may be used for storing temporary variables or other intermediate information during execution of instructions to be executed by processor 504. Such instructions, when stored in non-transitory computer-readable storage media accessible to processor 504, can render computer system 500 into a special-purpose machine that is customized to perform the operations specified in the instructions.

Computer system 500 further includes non-volatile memory such as read only memory (ROM) 508 or other static storage devices coupled to I/O subsystem 502 for storing information and instructions for processor 504. The ROM 508 may include various forms of programmable ROM (PROM) such as erasable PROM (EPROM) or electrically erasable PROM (EEPROM). A unit of persistent storage 510 may include various forms of non-volatile RAM (NVRAM), such as FLASH memory, or solid-state storage, magnetic disk, or optical disk such as CD-ROM or DVD-ROM and may be coupled to I/O subsystem 502 for storing information and instructions. Storage 510 is an example of a non-transitory computer-readable medium that may be used to store instructions and data which when executed by the processor 504 cause performing computer-implemented methods to execute the techniques herein.

The instructions in memory 506, ROM 508 or storage 510 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 500 may be coupled via I/O subsystem 502 to at least one output device 512. In an embodiment, output device 512 is a digital computer display. Examples of a display that may be used in various embodiments include a touch screen display or a light-emitting diode (LED) display or a liquid crystal display (LCD) or an e-paper display. Computer system 500 may include other type(s) of output devices 512, alternatively or in addition to a display device. Examples of other output devices 512 include printers, ticket printers, plotters, projectors, sound cards or video cards, speakers, buzzers or piezoelectric devices or other audible devices, lamps or LED or LCD indicators, haptic devices, actuators, or servos.

At least one input device 514 is coupled to I/O subsystem 502 for communicating signals, data, command selections or gestures to processor 504. Examples of input devices 514 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 516, which may perform cursor control or other automated control functions such as navigation in a graphical interface on a display screen, alternatively or in addition to input functions. Control device 516 may be a touchpad, a mouse, a trackball, or cursor direction keys for communicating direction information and command selections to processor 504 and for controlling cursor movement on a display. The control device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 514 may include a combination of multiple different input devices, such as a video camera and a depth sensor.

In another embodiment, computer system 500 may comprise an internet of things (IoT) device in which one or more of the output device 512, input device 514, and control device 516 are omitted. Or, in such an embodiment, the input device 514 may comprise one or more cameras, motion detectors, thermometers, microphones, seismic detectors, other sensors or detectors, measurement devices or encoders and the output device 512 may comprise a special-purpose display such as a single-line LED or LCD display, one or more indicators, a display panel, a meter, a valve, a solenoid, an actuator or a servo.

When computer system 500 is a mobile computing device, input device 514 may comprise a global positioning system (GPS) receiver coupled to a GPS module that is capable of triangulating to a plurality of GPS satellites, determining and generating geo-location or position data such as latitude-longitude values for a geophysical location of the computer system 500. Output device 512 may include hardware, software, firmware and interfaces for generating position reporting packets, notifications, pulse or heartbeat signals, or other recurring data transmissions that specify a position of the computer system 500, alone or in combination with other application-specific data, directed toward host 524 or server 530.

Computer system 500 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 500 in response to processor 504 executing at least one sequence of at least one instruction contained in main memory 506. Such instructions may be read into main memory 506 from another storage medium, such as storage 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 510. Volatile media includes dynamic memory, such as memory 506. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 502. Transmission media can also take the form of acoustic or light waves, such as those generated during radio-wave and infra-red data communications.

Various forms of media may be involved in carrying at least one sequence of at least one instruction to processor 504 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 500 can receive the data on the communication link and convert the data to a format that can be read by computer system 500. For instance, a receiver such as a radio frequency antenna or an infrared detector can receive the data carried in a wireless or optical signal and appropriate circuitry can provide the data to I/O subsystem 502 such as by placing the data on a bus. I/O subsystem 502 carries the data to memory 506, from which processor 504 retrieves and executes the instructions. The instructions received by memory 506 may optionally be stored on storage 510 either before or after execution by processor 504.

Computer system 500 also includes a communication interface 518 coupled to the bus on I/O subsystem 502. Communication interface 518 provides two-way data communication coupling to network link(s) 520 that are directly or indirectly connected to at least one communication network, such as a network 522 or a public or private cloud on the Internet. For example, communication interface 518 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 522 broadly represents a local area network (LAN), wide-area network (WAN), campus network, internetwork, or any combination thereof. Communication interface 518 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 518 sends and receives electrical, electromagnetic, or optical signals over signal paths that carry digital data streams representing various types of information.

Network link 520 typically provides electrical, electromagnetic, or optical data communication directly or through at least one network to other data devices, using, for example, satellite, cellular, Wi-Fi, or BLUETOOTH technology. For example, network link 520 may provide a connection through a network 522 to a host computer 524.

Furthermore, network link 520 may provide a connection through network 522 to other computing devices via internetworking devices and/or computers that are operated by an Internet Service Provider (ISP) 526. ISP 526 provides data communication services through a world-wide packet data communication network represented as internet 528. Server computer 530 may be coupled to internet 528. Server 530 broadly represents any computer, data center, virtual machine, or virtual computing instance with or without a hypervisor, or computer executing a containerized program system such as DOCKER or KUBERNETES. Server 530 may represent an electronic digital service that is implemented using more than one computer or instance and that is accessed and used by transmitting web services requests, uniform resource locator (URL) strings with parameters in HTTP payloads, API calls, app services calls, or other service calls. Computer system 500 and server 530 may form elements of a distributed computing system that includes other computers, a processing cluster, server farm or other organization of computers that cooperate to perform tasks or execute applications or services. Server 530 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 530 may comprise a web application server that hosts a presentation layer, application layer and data storage layer such as a relational database system using structured query language (SQL) or no SQL, an object store, a graph database, a flat file system or other data storage.

Computer system 500 can send messages and receive data and instructions, including program code, through the network(s), network link 520 and communication interface 518. In the Internet example, a server 530 might transmit a requested code for an application program through Internet 528, ISP 526, local network 522 and communication interface 518. The received code may be executed by processor 504 as it is received, and/or stored in storage 510, or other non-volatile storage for later execution.

The execution of instructions as described in this section may implement a process in the form of an instance of a computer program that is being executed and consisting of program code and its current activity. Depending on the operating system (OS), a process may be composed of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 504. While each processor 504 or core of the processor executes a single task at a time, computer system 500 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

12.0 Software Overview

FIG. 6 is a block diagram of a basic software system 600 that may be employed for controlling the operation of computing device 500. Software system 600 and its components, including their connections, relationships, and functions, is meant to be exemplary only, and not meant to limit implementations of the example embodiment(s). Other software systems suitable for implementing the example embodiment(s) may have different components, including components with different connections, relationships, and functions.

Software system 600 is provided for directing the operation of computing device 500. Software system 600, which may be stored in system memory (RAM) 506 and on fixed storage (e.g., hard disk or flash memory) 510, includes a kernel or operating system (OS) 610.

The OS 610 manages low-level aspects of computer operation, including managing execution of processes, memory allocation, file input and output (I/O), and device I/O. One or more application programs, represented as 602A, 602B, 602C . . . 602N (collectively, application(s) 602), may be “loaded” (e.g., transferred from fixed storage 510 into memory 506) for execution by the system 600. The applications or other software intended for use on device 600 may also be stored as a set of downloadable computer-executable instructions, for example, for downloading and installation from an Internet location (e.g., a Web server, an app store, or other online service).

Software system 600 includes a graphical user interface (GUI) 615, for receiving user commands and data in a graphical (e.g., “point-and-click” or “touch gesture”) fashion. These inputs, in turn, may be acted upon by the system 600 in accordance with instructions from operating system 610 and/or application(s) 602. The GUI 615 also serves to display the results of operation from the OS 610 and application(s) 602, whereupon the user may supply additional inputs or terminate the session (e.g., log off).

OS 610 can execute directly on the bare hardware 620 (e.g., processor(s) 504) of device 500. Alternatively, a hypervisor or virtual machine monitor (VMM) 630 may be interposed between the bare hardware 620 and the OS 610. In this configuration, VMM 630 acts as a software “cushion” or virtualization layer between the OS 610 and the bare hardware 620 of the device 500.

VMM 630 instantiates and runs one or more virtual machine instances (“guest machines”). Each guest machine comprises a “guest” operating system, such as OS 610, and one or more applications, such as application(s) 602, designed to execute on the guest operating system. The VMM 630 presents the guest operating systems with a virtual operating platform and manages the execution of the guest operating systems.

In some instances, the VMM 630 may allow a guest operating system to run as if it is running directly on the bare hardware 620 of device 500. In these instances, the same version of the guest operating system configured to execute on the bare hardware 620 directly may also execute on VMM 630 without modification or reconfiguration. In other words, VMM 630 may provide full hardware and CPU virtualization to a guest operating system in some instances.

In other instances, a guest operating system may be specially designed or configured to execute on VMM 630 for efficiency. In these instances, the guest operating system is “aware” that it executes on a virtual machine monitor. In other words, VMM 630 may provide para-virtualization to a guest operating system in some instances.

The above-described basic computer hardware and software is presented for purpose of illustrating the basic underlying computer components that may be employed for implementing the example embodiment(s). The example embodiment(s), however, are not necessarily limited to any particular computing environment or computing device configuration. Instead, the example embodiment(s) may be implemented in any type of system architecture or processing environment that one skilled in the art, in light of this disclosure, would understand as capable of supporting the features and functions of the example embodiment(s) presented herein.

13.0 Other Aspects of Disclosure

Embodiments of method and/or composition described here can include every combination and permutation of the various method processes and the various composition components, including any variants (e.g., embodiments, variations, examples, specific examples, figures, etc.), where portions of the method processes described herein can be performed asynchronously (e.g., sequentially), concurrently (e.g., in parallel), or in any other suitable order by and/or using one or more instances, elements, components of, and/or other aspects of the composition and/or other entities described herein, and where components can be additionally or alternatively combined, aggregated, excluded, and/or used.

As a person skilled in the art will recognize from the previous detailed description and from the figures and claims, modifications and changes can be made to the composition, method, and/or variants without departing from the scope defined in the following claims.

What is claimed is:

1. A virus-inhibiting amount of a pharmaceutical composition for molecular pattern recognition based targeting and activating an innate immune response, comprising:
a lectin component that binds to one or more glycosylated pathogens and that, upon binding, acts as an opsonin; and
a pharmaceutical component including at least one of a pharmaceutically-acceptable carrier and excipient;
wherein the composition is administered orally, intravenously, nasally, or inhaled; and
wherein the lectin component includes at least one of Allium porrum agglutinin, Urtica dioica agglutinin, Nicotiana tabacum agglutinin, and mistletoe lectin II.

2. The composition of claim 1, wherein the lectin component comprises at least one of a manufactured mannose binding lectin and a recombinant mannose binding lectin.

3. The composition of claim 1, wherein the lectin component comprises a natural lectin extracted or isolated from a plant.

4. The composition of claim 1, wherein the lectin component comprises a first lectin having a trimeric molecular structure and a second lectin having a tetrameric molecular structure, a dimeric molecular structure, or a monomeric molecular structure.

5. The composition of claim 4, wherein the lectin component comprises a third lectin such that the first, second, and third lectins have at least three of four molecular structures, wherein the four molecular structures are monomeric, dimeric, trimeric, and tetrameric.

6. The composition of claim 1, further comprising a flavonoid component including at least a flavonol or flavanol that binds to a main protease for the one or more glycosylated pathogens.

7. The composition of claim 6, wherein the at least one flavonoid comprises at least one of kaempferol, quercetin, EGCG, luteolin, cinnamtannin b1, hesperetin, and myricetin.

8. The composition of claim 1 wherein an amount of the lectin component in the composition for a single dose comprises at least one half of 1/7th the amount of a lectin necessary to reach an EC50 level of the lectin and less than 3 times the amount of lectin necessary to reach the EC50 level of the lectin for at least one of the glycosylated pathogens.

9. The composition of claim 8, wherein an amount of the lectin component in the composition for a single dose ranges between 160 μg and 11,675 μg of Allium porrum agglutinin.

10. The composition of claim 1 wherein an amount of the flavonoid component in the composition for a single dose comprises at least one half of one tenth the amount of a flavonoid necessary to reach an IC50 level of the flavonoid and less than 20 times the amount of lectin necessary to reach the IC50 level of the flavonoid for at least one of the glycosylated pathogens.

11. The composition of claim 10, wherein an amount of the flavonoid component in the composition for a single dose includes at least one of:
an amount ranging between approximately 3 mg to approximately 12.2 mg of kaempferol;
an amount ranging between approximately 1 mg to 4 mg of quercetin; and
an amount ranging between approximately 0.19 mg and 756 mg of EGCG.

12. The composition of claim 1, further comprising a supportive component that facilitates binding of the lectin component to the one or more glycosylated pathogens, wherein the support component comprises calcium.

13. The composition of claim 12, wherein an amount of the supportive component in the composition for a single dose ranges between at least 14.75 mg and 625 mg.

14. The composition of claim 1, wherein the pharmaceutical component comprises saline.

15. A virus-inhibiting amount of a pharmaceutical composition for pattern recognition based targeting and activating an innate immune response, comprising:
a lectin component that binds to one or more glycosylated pathogens and that, upon binding, acts as an opsonin and wherein the lectin component comprises at least one of a manufactured mannose binding lectin and a recombinant mannose binding lectin;
a flavonoid component comprising at least one flavonol or flavanol that inhibits the function of a main protease of the one or more glycosylated pathogens; and
a pharmaceutical component including at least one of a pharmaceutically-acceptable carrier and excipient.

16. The composition of claim 15, wherein the lectin component includes at least one of Allium porrum agglutinin, Urtica dioica agglutinin, Nicotiana tabacum agglutinin, and mistletoe lectin II.

17. The composition of claim 15, wherein the lectin component comprises a first lectin having a trimeric molecular structure and a second lectin having a tetrameric molecular structure, a dimeric molecular structure, or a monomeric molecular structure.

18. The composition of claim 15, wherein the flavonoid component comprises at least one of kaempferol, quercetin, EGCG, luteolin, cinnamtannin b1, hesperetin, and myricetin.

19. A method of treating SARS coronavirus infections, comprising:
obtaining a virus-inhibiting amount of a pharmaceutical composition for molecular pattern recognition based targeting and activating an innate immune response, the composition comprising a lectin component that binds to one or more glycosylated pathogens and that, upon binding, acts as an opsonin, wherein the lectin component includes a therapeutically effective amount of at least one of Allium porrum agglutinin, Urtica dioica agglutinin, Nicotiana tabacum agglutinin, and mistletoe lectin II; and
administering the composition to a subject.

20. The method of claim 19, wherein the pharmaceutical composition further comprises a flavonoid component including at least a flavonol or flavanol that binds to a main protease for the one or more glycosylated pathogens.
